(19) United States
(12) Patent Application Publication
(10) Pub. No.: US 2014/0081305 A1
(43) Pub. Date: Mar. 20, 2014

(54) Hollow Organ Coring Tool with Collapsing Anvil and Method of Use

(71) Applicant: Indian Wells Medical, Inc., Laguna Beach, CA (US)

(72) Inventors: Eugene M. Breznock, Winters, CA (US); Jay A. Lenker, Laguna Beach, CA (US)

(73) Assignee: Indian Wells Medical, Inc., Laguna Beach, CA (US)

(21) Appl. No.: 14/020,618

(22) Filed: Sep. 6, 2013

Related U.S. Application Data
(60) Provisional application No. 61/697,385, filed on Sep. 6, 2012.

(51) Int. Cl. A61B 17/3205

(52) U.S. Cl. CPC A61B 17/32053
USPC 606/184

(57) ABSTRACT

A coring tool for creating a hole in a body vessel or hollow organ. The coring tool includes an expandable anvil against which the cutter can be advanced following passage of the collapsed anvil through the tissue to be excised and subsequent expansion of the anvil.

This application claims priority to U.S. Provisional Application 61/697,385 filed Sep. 6, 2012, the contents of which are hereby incorporated by reference.

### FIELD OF THE INVENTION

The inventions described below relate the field of hollow organ coring tools.

### BACKGROUND OF THE INVENTION

During surgical procedures such as placement of a ventricular assist device, blood vessel anastomosis, aortotomy, gastrotomy, enterotomy, or access to other hollow organs and vessels, it is useful to have a specialized coring tool to create a circular opening or fenestration in the wall of the vessel or organ. Our prior patent, Breznock, Method and Apparatus for Trephinating Body Vessels and Hollow Organ Walls, U.S. Pat. No. 6,863,677 (Mar. 8, 2005) disclosed an improved coring tool with an anvil surface opposing the circular cutter. The anvil provided a surface against with the cutter could act while cutting through an organ wall, leading to a cleaner cut. The anvil was slightly larger in diameter than the cutter, and, since it had to be forced through a pilot incision in the organ wall, could lead to tearing of the organ wall. This makes subsequent anastomosis or suturing of tubes to the organ wall difficult.

### SUMMARY OF THE INVENTIONS

The devices and methods described below provide for easier insertion of the anvil portion of the Breznock coring tool. An anvil is disposed at the distal tip of the device. The anvil, which is inserted into a hollow organ prior to cutting, is reconfigurable from a low profile configuration for insertion through an incision and a high profile configuration which presents an anvil surface in apposition to the cutter of the coring tool.

In some embodiments, the anvil is collapsible to a small diameter configuration and expandable to a large diameter configuration. In the small diameter configuration, the tip of the coring tool can be inserted through a small incision in the organ, into the inside of an organ (typically the heart or large artery). After insertion, the tip can be expanded to provide an anvil upon which the cutter may act. In other embodiments, the anvil is reconfigurable to rotate relative to the long axis of the device, to dispose the edge of flat ring facing distally for insertion, and then to rotate to position the flat ring perpendicular to the long axis of the device to present an anvil surface which faces proximally and apposes the cutter.

### BRIEF DESCRIPTION OF THE DRAWINGS

FIG. 1A illustrates a side view of a coring tool incorporating an expandable anvil in a molly-bolt configuration in its first, collapsed configuration.

FIG. 1B illustrates a side view of a coring tool incorporating an expandable anvil in a Molly-bolt configuration in its second, expanded configuration.

FIG. 2 illustrates the coring tool applied to the apex of the ventricle of the heart prior to advancing the cutting blade.

FIG. 3 illustrates the coring tool after the blade has been advanced through the apex of the ventricular wall of the heart.

FIG. 4 illustrates the ventricular wall after removal of the coring tool and the excised tissue.

FIG. 5A illustrates a coring tool incorporating an expandable anvil in a mesh configuration in its first, collapsed configuration.

FIG. 5B illustrates a coring tool incorporating an expandable anvil in a mesh configuration in its second, expanded configuration.

FIG. 6A illustrates a coring tool incorporating an expandable anvil in a rotating plate configuration in its first, collapsed configuration.

FIG. 6B illustrates a coring tool incorporating an expandable anvil in a rotating plate configuration in its second, expanded configuration.

FIGS. 7A, 7B and 7C illustrates a coring tool incorporating an expandable anvil in a hinged, rotating plate configuration.

FIGS. 8A and 8B illustrate a coring tool incorporating an expandable anvil in a molly-bolt configuration further comprising a connecting web.

FIGS. 9A and 9B illustrate a coring tool incorporating an expandable anvil in an umbrella configuration.

FIGS. 10A and 10B illustrates a side view of a coring tool incorporating an expandable anvil in a cone configuration.

FIGS. 11A and 11B illustrates a coring tool incorporating an expandable anvil in a balloon configuration.

### DETAILED DESCRIPTION OF THE PREFERRED EMBODIMENTS

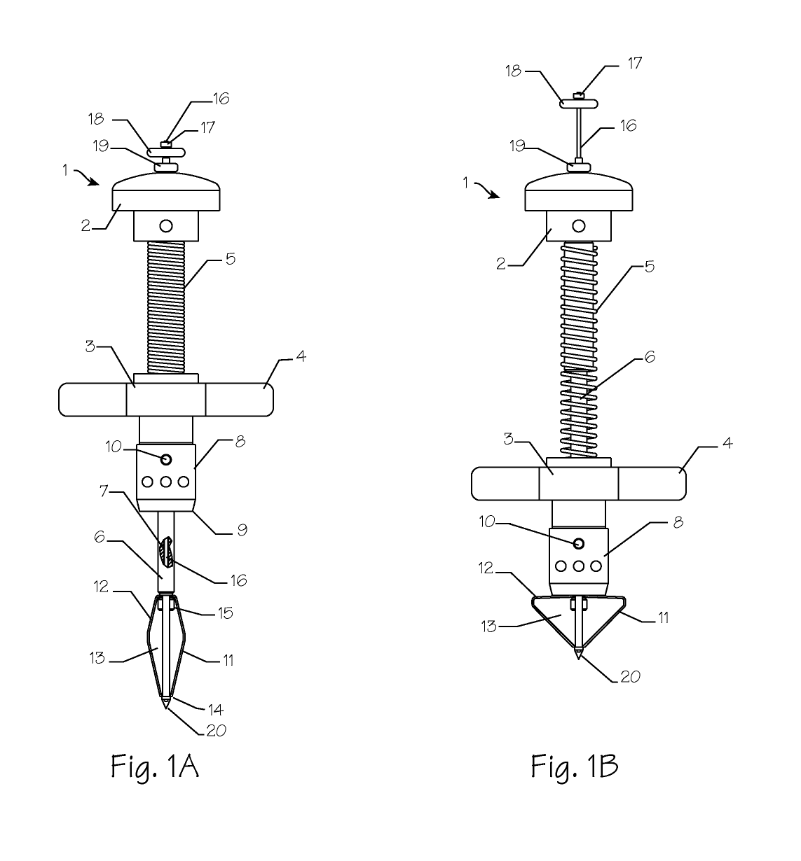

FIG. 1A illustrates a side view of a coring tool 1 incorporating an expandable anvil in a molly-bolt configuration in its first, collapsed configuration. The coring tool comprises the knob 2, a handle 3 further comprising the grips or wings 4, the spring 5, the core shaft 6 further comprising an inner lumen 7, the cutter 8 further comprising the cutter distal edge 9, the cutter attachment fastener 10, an expandable anvil 11 further comprising a plurality of anvil legs or struts 12 separated by a plurality of slots 13, a distal strut attachment 14, proximal strut attachment 15, an anvil control shaft 16, a control shaft proximal fastener 17, an anvil control shaft knob 18, and an anvil control shaft lock 19. The anvil struts are joined at their distal ends to the distal end of the anvil control shaft 16 by the distal strut attachment 14 and are longitudinally fixed to the anvil control shaft 16 (they may be rotationally fixed as well). The anvil struts are joined at their proximal ends to the distal end of the core shaft 6 and are longitudinally fixed to the core shaft (they may be rotationally fixed as well). Proximal translation of the anvil control shaft 16 relative to the core shaft 6 forces the sleeve to expand as illustrated in FIG. 1B. The coring tool is shown in FIG. 1A with the anvil in its first collapsed configuration and the cutter 8 retracted proximally.

The anvil control shaft 16 is slidably disposed through the lumen 7 in the core shaft 6 and is constrained to move only axially within the lumen 7. The core shaft 6 is not visible under the spring 5 because the spring 5 is fully compressed with its coils adjacent to each other. The spring 5 biases the handle 3 and the cutter 8 distally toward the anvil 11 with a controlled amount of force. The core shaft 6 is affixed, at its proximal end, to the knob 2 and, at its distal end, to the proximal end of the anvil 11. The handle 3 is constrained to rotate about the axis of the core shaft 6 and can slidably move thereupon. The handle 3 is affixed to the cutter 8 by the fastener 10 but can also be bonded, welded, insert molded, press-fit, snapped on, or the like. The cutter 8 rotates with the handle 3, relative to the anvil 11.

FIG. 1B illustrates the coring tool 1, with the anvil 11 in its second, expanded configuration and the cutter 8 advanced distally, comprising the knob 2, a handle 3 further comprising the grips or wings 4, the spring 5, the core shaft 6, the cutter 8 further comprising the cutter distal edge 9, the cutter attachment fastener 10, the expandable molly-bolt anvil 11 further comprising the plurality of anvil struts 12, the distal strut attachment 14, the anvil control shaft 16, the control shaft proximal fastener 17, the anvil control shaft knob 18, and the anvil control shaft lock 19.

The anvil control shaft knob 18 is affixed to the anvil control shaft 16 by the control shaft proximal fastener 17. The anvil control shaft 16 is affixed to the distal end of the molly-bolt anvil 11 by the distal strut attachment 14. The proximal end of the molly-bolt anvil 11 is affixed to the distal end of the core shaft 6. The anvil control shaft 16 has been retracted proximally by forcible traction on the knob 18 and the anvil control lock 19 has been engaged to be releasably affixed to the shaft 16. The control lock 19 prevents proximal movement of the shaft 16 through the central lumen of the core shaft 6. The anvil struts 12 are bent outward in a central region and the proximal portions of the anvil struts 12 are disposed with a proximally facing surface, opposing the cutting edge of the cutter 8, to form a surface against which the cutter 8 can press. In this embodiment, it is beneficial to rotate the cutter 8 and counter-rotate the anvil 11 to provide complete cutting of tissue trapped between the cutter and the anvil. Though the core shaft and anvil control shaft are depicted as coaxial, with the anvil control shaft disposed coaxially within the core shaft, the two shaft need not be coaxial, so long as they are longitudinally translatable relative to each other. The distal tip of the device can be provided with a penetrating tip 20, which is sharp enough to penetrate body tissue without a first making a pilot incision in the tissue of the hollow body organ.

The anvil 11 depicted in FIGS. 1A and 1B is a molly-bolt type expanding sleeve fabricated from malleable or spring materials. The anvil 11 can be fabricated from tubing into which longitudinal slots 13 are cut to form a plurality of legs or struts 12 biased bend outwardly at a point between the distal and proximal end of the struts. The struts, in the small diameter insertion configuration, are roughly parallel to the control shaft. The struts 12 expand radially apart as the anvil 11 becomes compressed longitudinally causing lateral, diametric, or radial expansion of the struts 12, such that proximally portions of the struts present a proximally facing surface, in apposition to the cutter. Preferably, the proximal end of the struts 12 are bent into a roughly horizontal or laterally projecting arm against which the cutter edge 9 can impinge but they need only expand sufficiently to present an anvil surface of about the same diameter, and preferably slightly larger than, the cutter diameter. The proximal portions, in the large diameter configuration are divergent from, and roughly perpendicular to the anvil control shaft 16. The anvil 11 can comprise about two to about 20 struts 12 with an equivalent number of slots 13. As shown in the figures, the length of the proximal struts 12 is sufficient that the outermost diameter of the expanded anvil 11 is greater than the diameter of the sharp edge 9 of the cutter. The preferred diameter can range from about 1 mm to about 30 mm depending on the design of the cutter 8.

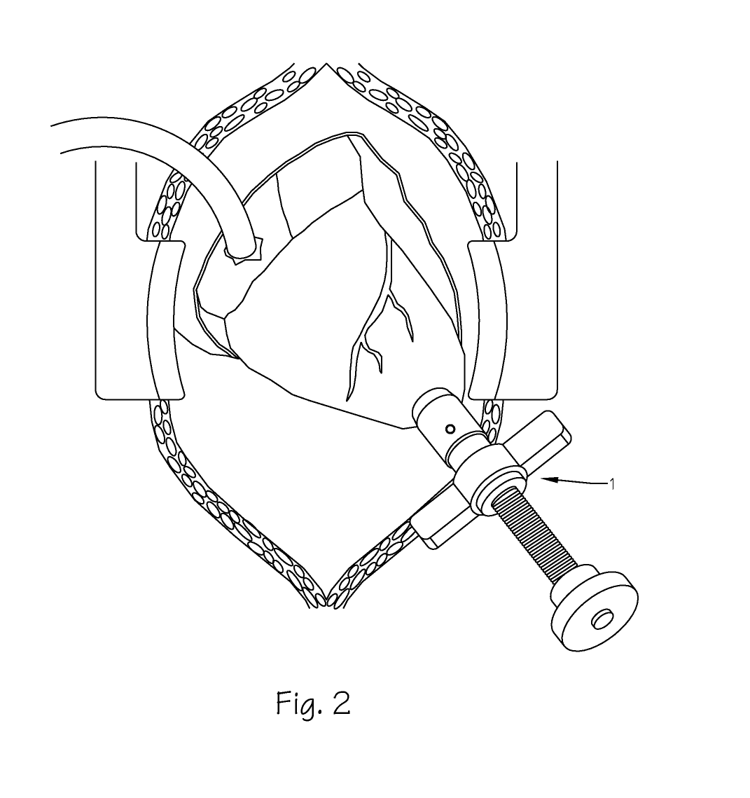

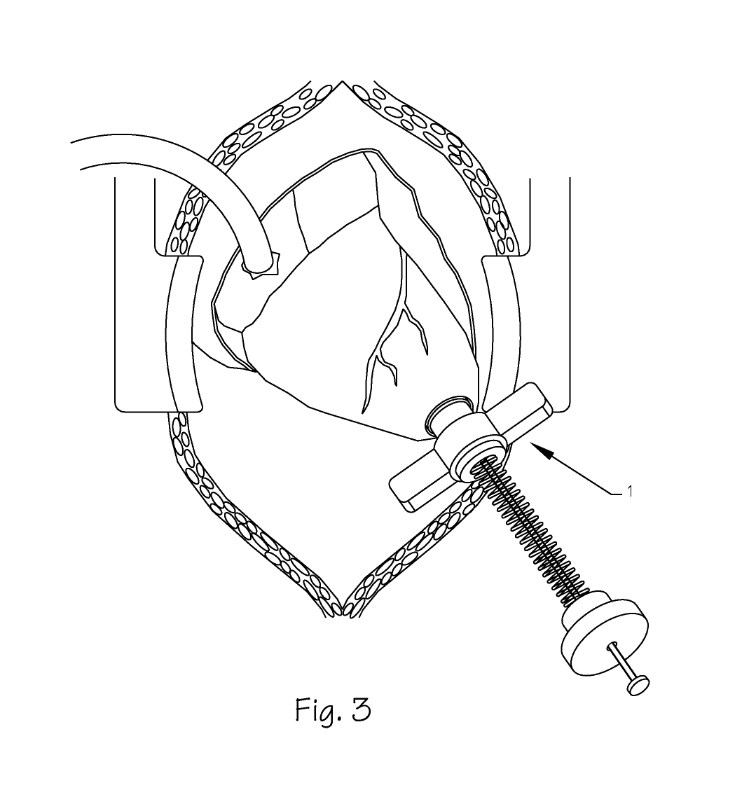

FIGS. 2, 3, and 4, illustrate the procedure for hollow organ coring accomplished with the coring tool with a collapsible anvil. A surgeon first makes a small incision at the desired penetration location using a sharp surgical instrument such as a scalpel. The cutter 8 is retracted by manually withdrawing the handle 3 and wings 4 proximally toward the knob 2. The spring 5 is compressed when retracting the handle 3 and cutter 8. The surgeon advances the anvil assembly, in its small diameter configuration, into the incision until the anvil 11 has passed beyond the interior surface of the hollow organ or vessel. (As an alternative, if the coring tool is fitted with the penetrating tip 20, the distal end of the device can be pushed through organ wall without first cutting a pilot incision.) The surgeon then operates the anvil control shaft 16 to expand the anvil, to the configuration shown in FIG. 1B. The surgeon may then release the handle to position the cutter 8 against the exterior of the hollow organ as shown in FIG. 2. Once the position has been confirmed or adjusted, the surgeon rotates the handle 3 to initiate cutting of the tissue by the cylindrical cutter 8. The spring urges the cutter into the tissue, and the surgeon may also exert force distally to urge the cutter into the tissue and toward the anvil. As shown in FIG. 3, the handle 3 and cutter 8 are rotated until full penetration of the hollow organ has occurred, under force of the spring 5.

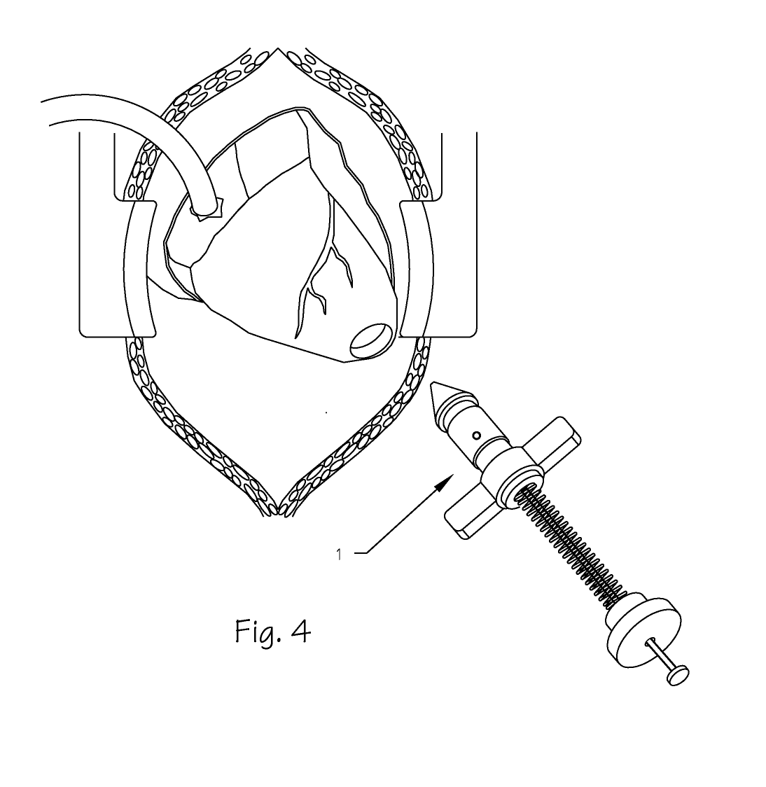

The coring tool 1 is next withdrawn proximally, removing the cored-out piece of tissue from the organ as shown in FIG. 4.

The remaining figures illustrate alternative embodiment of the coring tool, with alternative configurations of the anvil assembly with may be configured to present a low profile for insertion through a small incision and then, after insertion, may be reconfigured to present an anvil surface opposing the cutter.

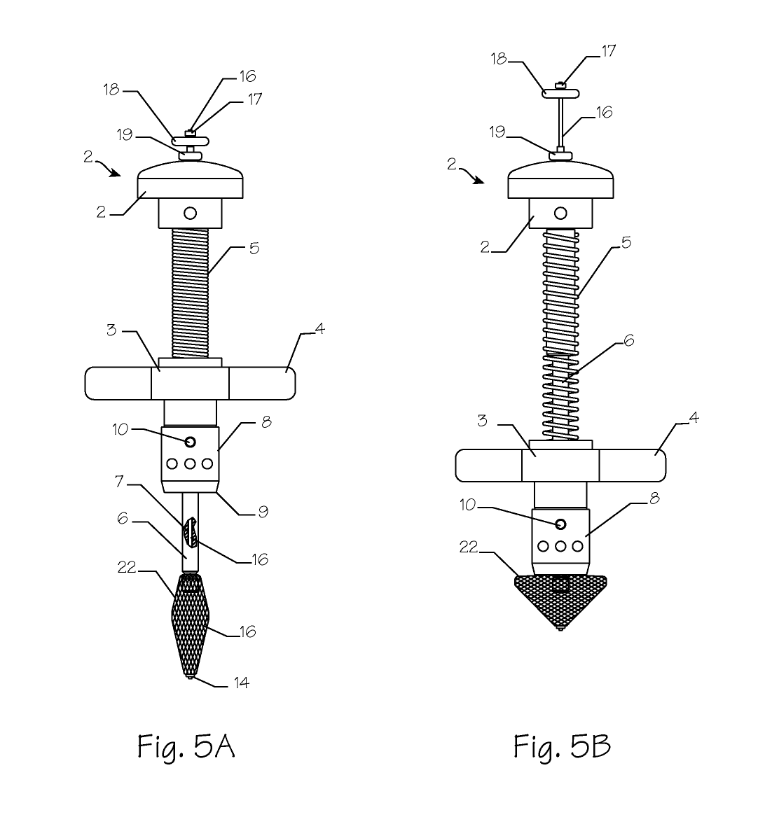

FIGS. 5A and 5B show a coring tool with an expandable mesh anvil. FIG. 5A illustrates a coring tool 21 with the cutter 8 retracted proximally and the anvil collapsed in its first, smaller diameter configuration. The coring tool 21 comprises the knob 2, the handle 3 further comprising the grips or wings 4, the spring 5, the core shaft 6 further comprising the inner lumen 7, the cutter 8 further comprising the cutter distal edge 9, the cutter attachment fastener 10, and an expandable mesh anvil 22. The mesh anvil comprises a mesh basket. The mesh comprises a plurality of wires woven into a mesh. The device also includes the distal anvil attachment 14, an anvil control shaft 16, the control shaft proximal fastener 17, the anvil control shaft knob 18, and the anvil control shaft lock 19. The control shaft is fixed to the distal apex of the mesh anvil at the distal anvil attachment, so that proximal motion of the anvil control shaft forces the mesh to expand radially and contract longitudinally.

The mesh anvil 22 is affixed, at its proximal end, to the core shaft 6 and, at its distal end, to the anvil control shaft 16 which is slidably disposed within the lumen 7 of the core shaft 6. The anvil 22 comprises a braid, mesh, or diamond pattern of metal or polymer that expands radially upon axial compression of the proximal end of the mesh relative to the distal end of the mesh. The mesh wires of the anvil can comprise metals such as, but not limited to, tantalum, platinum, gold, titanium, stainless steel, cobalt nickel alloy, n nitinol, or the like. The mesh wires can also comprise polymers such as, but not limited to, acetal copolymer, acetal homopolymer, polyester (PET), polyethylene naphthalate (PEN), polyamide, polyimide, or the like. The anvil wires can also comprise polymer coated metals. These same materials can be used for the struts 12 of the molly-bolt anvil 11 in FIGS. 1A and 1B.

FIG. 5B illustrates the coring tool 21, with the anvil 22 in its second, expanded configuration and the cutter 8 advanced distally. The spring 5 has expanded forcing the cutter 8 to move distally against the anvil 22. The anvil 22 has expanded radially, and collapsed longitudinally, as the anvil control shaft 16 has been pulled proximally. The lock nut 19 can be engaged to maintain the position of the anvil control shaft 16 and, by result, the anvil 22. The shape of the anvil 22 can be tailored by pre-shaping the wires of the mesh. The illustrated anvil 22 comprises a conical shape with a substantially flat proximally facing shoulder that is oriented toward the cutter 8. The small spaces in the mesh facilitate cutting of tissue between the anvil 22 and the cutter 8 without significantly rotating the anvil 22.

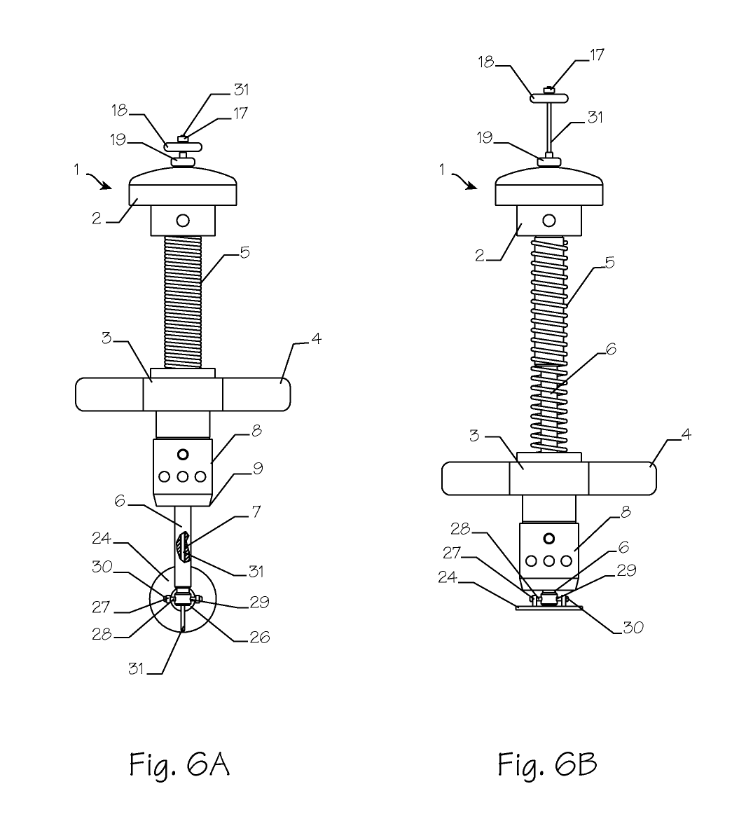

FIGS. 6A and 6B show a coring tool with an rotatable plate for an anvil. The anvil is reconfigurable from a low profile configuration relative to the transverse axis of the device to a high profile configuration relative to the transverse axis of the device. FIG. 6A illustrates a coring tool 23 and rotating plate anvil 24. The cutter 8 is retracted proximally and the anvil rotated in its first, lower profile insertion configuration. The coring tool 23 comprises the knob 2, the handle 3 further comprising the grips or wings 4, the spring 5, the core shaft 6 further comprising the inner lumen 7, the cutter 8 further comprising the cutter distal edge 9, and a rotating plate anvil 24. The rotating plate anvil comprises flat plate 25 with a central gap 26, a plurality of shaft supports 27, an axle 28, an axle bearing 29, a plurality of axle fasteners 30, an anvil lock shaft 31, the anvil lock shaft proximal fastener 17, the anvil lock shaft knob 18, and the anvil lock shaft lock 19.

The operation of the coring tool 23 is similar to other coring tools described herein except that the anvil 24 is a plate comprising a substantially flat side that is rotated approximately 90 degrees from its insertion orientation which is substantially parallel to the axis of the core shaft 6 (that is, the plane defined by the flat surface of the plate is parallel to the longitudinal axis of the device when the plate is rotated for insertion). The anvil 24 rotates about the axle bearing 29 which can comprise a hole oriented laterally within the distal end of the core shaft 6. The axle 28 is constrained from movement along its own longitudinal axis by the shaft supports 27, which are affixed to the anvil 24 by bonding, welding, integral molding, insert molding, or the like. The anvil lock shaft 31, when advanced distally, prevents rotation of the anvil 24 even with substantial forces exerted thereon. The anvil lock shaft 31 is affixed to the anvil lock shaft knob 18 at the proximal end of the coring tool 23 and proximal withdrawal of the anvil lock shaft knob 18 moves the anvil lock shaft 31i proximally to permit rotation of the anvil about its axle 28. The anvil 24 is eccentrically suspended (off center) from the axle 28 by the shaft supports 27 and rotates to present a flat side to the cutter 8 without any intervention other than proximal withdrawal of the anvil 24 against any tissue through which the anvil has been advanced. FIG. 6A illustrates the low profile configuration of the device.

FIG. 6B illustrates the coring tool of FIG. 6A with the anvil lock shaft 31 withdrawn proximally to unlock the anvil 24. The anvil 24, specifically the flat plate 25, has been rotated about its axle 28 and is oriented orthogonally or laterally to the axis of the core shaft 6 such that the flat surface of the plate is facing distally, in apposition to the cutter. FIG. 6B illustrates the high profile configuration of the device. The anvil is oriented transverse to the long axis of the device to present a flat surface in apposition to the cutter. The cutter 8 has been spring biased against the anvil 24.

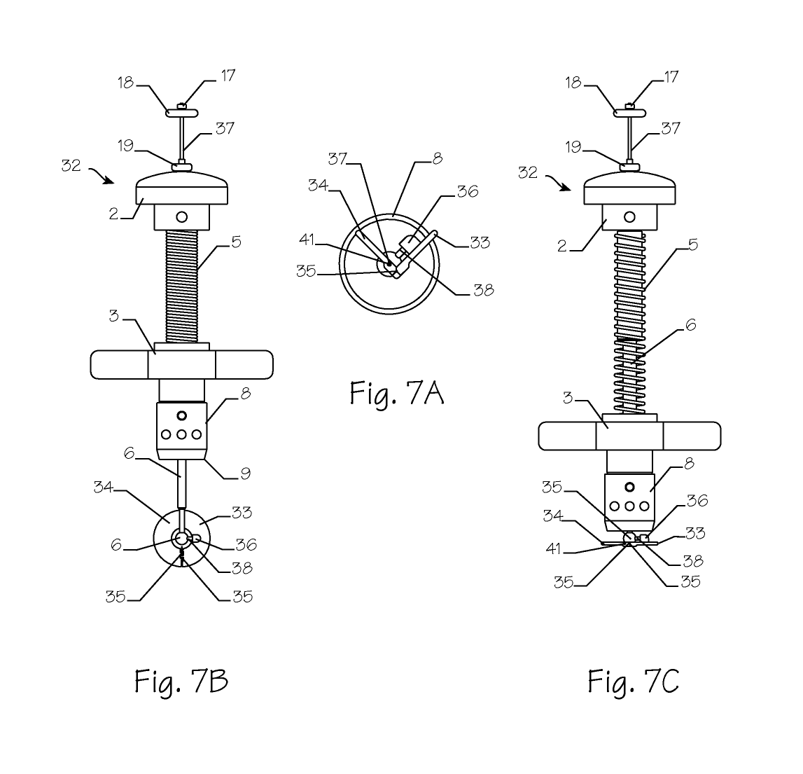

FIGS. 7A, 7B and 7C show a coring tool with a rotating and folding plate for an anvil. The plate rotates as in FIGS. 6A and 6B to present a smaller transverse profile, and also folds to reduce its maximum transverse dimension. FIG. 7B illustrates a folding, rotating hammer anvil for coring tool 32. The coring tool 32 comprises the cutter 8, two semi-circular anvil panels 33 and 34,which are joined together by a hinge 35. Anvil panel 33 is attached directly to the control shaft at axle mount 36 on the proximal surface of the anvil panel, while anvil panel 34 is rotatably attached, through the hinge, to the anvil panel 33. The device includes lock sleeve 37, an anvil lock shaft 31, an axle mount 36, an axle 38, a hinge 35, and a hinge pin 39.

The axle mount 36 s affixed to an upper surface of the attached anvil panel 33. A first end of the axle 38 is embedded within the axle mount 36 such that axial translation of the axle 38 is prohibited but rotational motion of the axle 38 relative to the axle mount 36 can occur. A second end of the axle 38 is constrained within the distal end of a anvil lock shaft 31 to be able to rotate about its longitudinal axis but the axle 38 cannot substantially move in the direction of its longitudinal axis. The attached anvil panel 33 and the hinge anvil panel 34 comprise integral hinge projections with a hole through them such that the hinge pin 39 maintains the hinge projections and their holes within coaxial alignment permitting rotational movement about the hinge pin 35 but linear movement along the direction of the axis of the hinge pin 35 is prohibited. The hinged anvil panel 34 is rotated roughly 90 degrees out of its operational plane to reduce its transverse or radial profile for tissue penetration (this is the low profile configuration). The hinged anvil panel 34 comprises the coring tool lock sleeve 37, which is a hole, lumen, or window formed through the hinged anvil panel 34. Extension of the anvil lock shaft 31 distally through the lock sleeve 37 prevents rotation of the hinged anvil panel 34 about its axis. The anvil lock shaft 31 is releasable by grasping a button and withdrawing the anvil lock shaft 31 in the proximal direction.

FIG. 7B illustrates a side view of the coring tool 32. The coring tool is illustrated with its anvil plates 34 and 33 unhinged to form a single surface or plane, and with the entire planar anvil subassembly rotated about 90 degrees from its insertion plane, laying in a plane parallel to the longitudinal axis of the device, which is orthogonal to the axis of the core shaft 6. The hinged anvil plate 34 has rotated to become substantially aligned with the attached anvil plate 33 which is rotatably affixed to the tilt axle 38 by means of the tilt axle retainer 40 which is affixed to the attached anvil plate 33. The tilt axle 38 is rotatably affixed to the distal end of the core shaft 6 and can rotate about its axis but not move along the axis of the tilt axle 38. The attached anvil plate 33 and the hinged anvil plate 34 can unhinge only enough to form a flat structure in a single plane due to bumpers or other projections or faces that prevent over-rotation. The control rod 37 is affixed at its proximal end to the anvil control shaft knob 18 and the control rod 37 is affixed at its distal end to a pin 31. The control rod 37 and the control pin 41 have been retracted proximally in FIG. 7B so that the hinged plate 34 can rotate to align with the plane of the attached anvil plate 33.

FIG. 7C illustrates the coring tool 32 following distal advancement of its cutter 8 and handle 3 toward the anvil plates 34 and 33 against which the cutter 8 impinges with few or no gaps in the contact area therebetween. The anvil plates 34 and 33 have swiveled about the axle 38 to their operational position in a plane orthogonal to the longitudinal axis of the coring tool 32. This is the high profile configuration. The swiveling about the axle 38 can be forced or it can be generated by the eccentric positioning of the plates 34 and 33 off center relative to the axle 38 and the axle retainer 40 since the axle retainer 40 is affixed to the proximal side of the attached anvil plate 33 and any force on the attached anvil plate 33 will swing it distal to the axle 38. At this point, rotation of the cutter 8 by turning the handle 3 will cause the cutter 8 to cut through tissue until it reaches the proximal side of the anvil plates 34 and 33. The cutter 8 inside diameter is greater than the radial positioning of the axle retainer or any other structures projecting proximally from the plates 34 and 33. The presence of these structures 40, 35, etc. does not substantially inhibit cutting the tissue plug because a small incision is already present in the tissue when the anvil components are advanced through the tissue. The tissue plug that is excised by this or any coring tool described herein can be removed from the patient along with the coring tool.

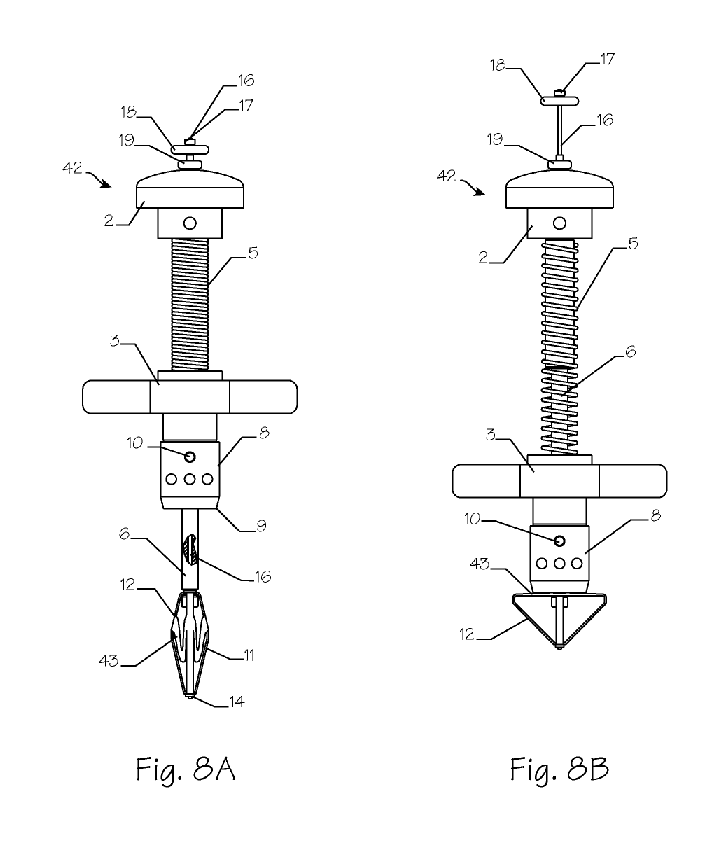

FIGS. 8A and 8B show a coring tool with an expanding sleeve which opens to present an anvil surface apposite the cutter. FIG. 8A illustrates a coring tool 42 with its anvil in a first, collapsed configuration, comprising a collapsing anvil in a molly-bolt configuration similar to that of FIGS. 1A and 1B, with many of the same components, except that a plurality of flexible or semi-flexible bands 43 are affixed to the struts 12. The cutter 8 is retracted proximally away from the anvil and the spring 5 is compressed. The bands 43 are affixed at one end to one strut 12, and at the other end, to an adjacent strut 12. The number of bands 43 preferably equals the number of gaps or spaces between the struts 12, which generally equals the number of struts 12. The band 43 is configured to pull tightly and fill in the space between the struts 12 such that the cutter distal edge 9 is in full contact with the cutter following expansion of the anvil struts 12. The number of bands 43 can range from about 2 to about 20. The band 43 can be fabricated from metals, polymers, polymer coated metals, or the like. Suitable polymeric materials include, but are not limited to, Nylon, Teflon, polyethylene, polypropylene, polyimide, polyamide, PEEK, and the like. Suitable metallic materials include, but are not limited to, stainless steel, tantalum, gold, platinum, titanium, nitinol, cobalt nickel alloy, or other suitable biocompatible metal. The structure of the bands 43 can comprise a solid band or woven, knitted, or braided fabrics, or the like. The bands 43 can be affixed to the struts 12 by fasteners, welding, adhesives, overmolding, insert molding, integral extension of strut coatings, and the like.

FIG. 8B illustrates the coring tool 42 of FIG. 8A with its anvil struts 12 expanded radially outward. The bands 43 are stretched along the upper edge that directly abuts the cutter 8 to form a substantially smooth, continuous surface against which the cutter 8 can cut tissue. In yet other embodiments, the bands 43 can be replaced by flaps (not shown) that project downward from the proximal edge of the struts 12 and fold outward in response to expansion of the struts 12 to which the flaps are affixed. The bands (not shown) can be affixed at the proximal end of the struts 12 and to a midpoint of the struts 12 such that they move with the struts 12 but are hinged or flexed such that they can conform to the changing shape of the anvil structure as it expands.

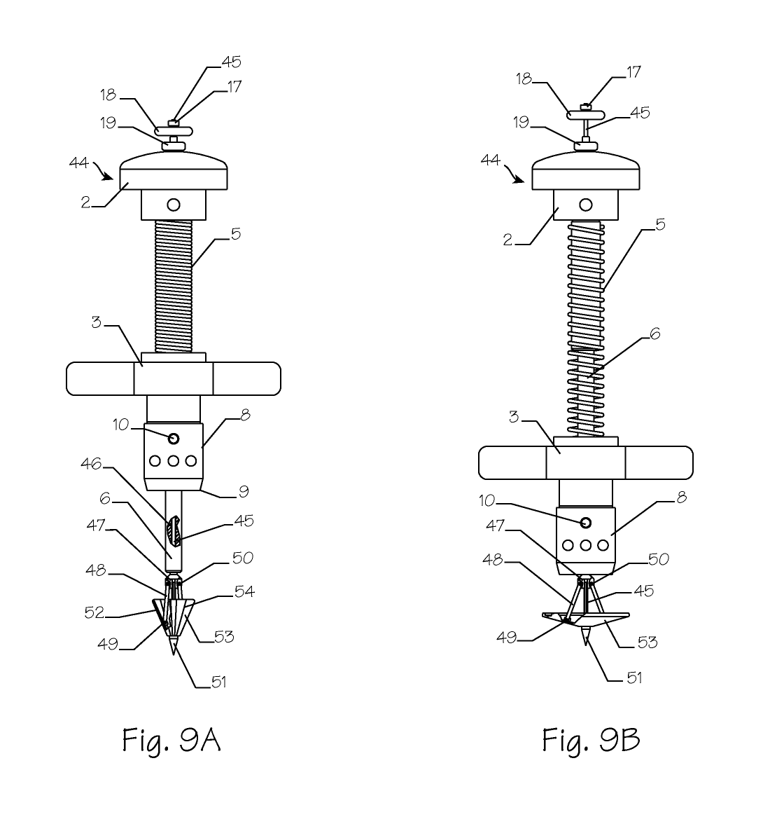

FIG. 9A illustrates a coring tool 44 with an umbrella anvil in a first radially smaller or collapsed configuration and with the cutter retracted proximally away from the anvil. The coring tool 44 comprises the knob 2, the spring 5, the handle 3, the cutter 8 further comprising the cutter distal edge 9 and the fastener 10, the core shaft 6, an umbrella control rod 45, the control linkage rod 18, the control linkage fastener 17, the control linkage lock 19, a control linkage lumen 46, a proximal umbrella base 47, a plurality of umbrella support struts 48, a plurality of umbrella distal support hinges 49, a plurality of umbrella proximal support hinges 50, a penetrating tip 51, a plurality of umbrella supports 52, an umbrella membrane 53 and a plurality of umbrella membrane folds 54.

The umbrella supports 52 are rotatably affixed to the umbrella supports 52 by the distal hinges 49. The umbrella supports 52 are affixed, at their proximal end, to the proximal umbrella base 47 by the proximal hinges 50. The umbrella membrane 53 is affixed to the umbrella supports 52, which comprise arms, rotatably affixed at their distal end to the penetrating tip 51 by hinges (not shown). The umbrella membrane 53 is preferably affixed to the proximal side of the umbrella supports 52 so that a substantially smooth, unbroken and substantially even surface can be presented to the cutter 8 when the umbrella membrane 53 is opened up as illustrated in FIG. 9B. The umbrella control rod 45 and its operating structures on the proximal end of the knob 2 are advanced distally to move the penetrating tip 51 and the distal end of the umbrella supports 52, to their maximum distal displacement.

The penetrating tip 51 can be blunt or sharp. The penetrating tip 51, can be used to create the initial incision or increase the size of the incision in a controlled fashion. The penetrating tip 51 in a blunt configuration can be used as a blunt dissection tool to permit advancement of the anvil through the tissue to be cut. This cutting penetrating tip 51, either sharp or blunt, can be configured for use on any of the coring tools described in this specification. In yet other embodiments, the cutting penetrating tip 51 can comprise a retraction mechanism (not shown) to permit an initial cut and then retract the penetrating tip 51 into a protective, atraumatic shroud such that further cutting is not performed by an integral sharp object 51.

The membrane 53 can comprise a solid band or woven, knitted, or braided fabrics, or the like. The membrane 53 can be affixed to the struts 52 by fasteners, welding, adhesives, over-molding, insert molding, integral extension of strut coatings, and the like.

FIG. 9B illustrates the coring tool 44 of FIG. 9A with the umbrella anvil membrane 53 opened or expanded to its operating position. The position of the strut 48 attachment to the support struts 52 at the distal hinges 49 is operationally smaller than the diameter of the cutter 8 so as not to impinge on or interfere with the cutter 8 and its travel into the anvil membrane 53. Additional supports can exist outside the diameter of the cutter 8 but the membrane 53 surface at the circle of cutter contact is beneficially substantially smooth, even, and unbroken. The umbrella control rod 45 is withdrawn proximally and the lock 19 is engaged to keep the umbrella control rod 45 in position relative to the core shaft 6.

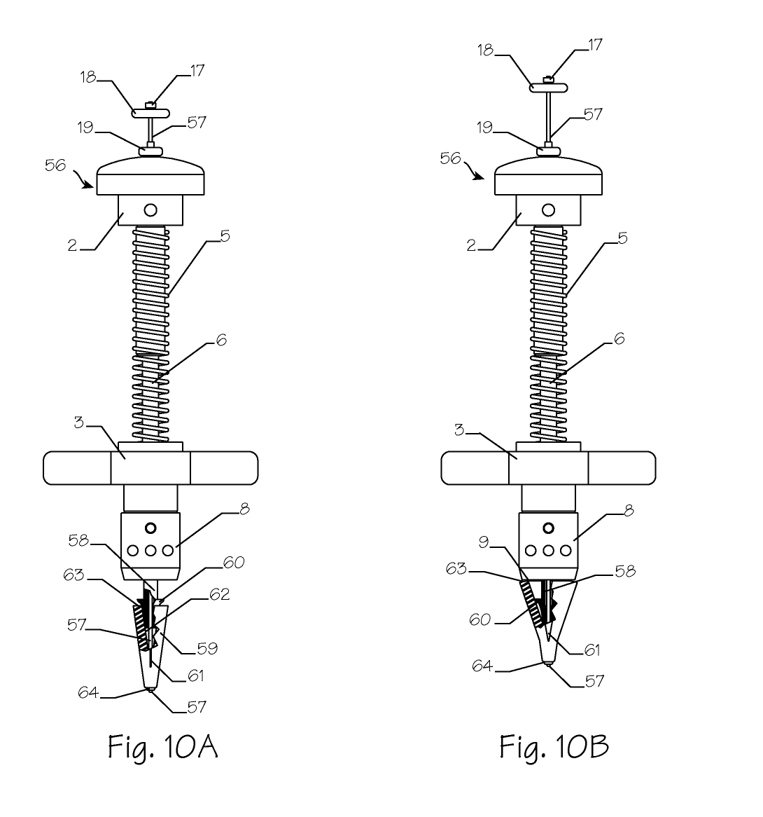

FIG. 10A illustrates a coring tool 56 comprising an expandable anvil in the form of an expandable cone. The coring tool 56 is illustrated with its cutter partially advanced toward the anvil, with the spring expanded, but with the anvil collapsed to its smallest cross-sectional configuration. The coring tool 56 comprises the knob 2, the spring 5, the core shaft 6, the handle 3, the cutter 8, a control shaft 57, the proximal control shaft fastener 17, the control rod knob 18, the control rod lock 19, the control rod proximal fastener 17, a cone core support 58, a cone anvil 59 and a cone spreader 60. The cone further comprising a plurality of slots or splits 61 oriented longitudinally with respect to the axis of the core shaft 6, a tapered inner surface 62, and a proximally facing anvil surface 63. It is longitudinally fixed to the control rod through distal control rod fastener 64, so that the control rod can be pulled proximally to force the cone proximally over the cone spreader, thereby forcing the cone base to widen to widen the anvil surface to approximate the diameter of the cutter 8.

The cone anvil 59 can be fabricated from the same materials as used for the anvils of coring tools described in the previous figures. The cutting surface 63 can be the same or a different material as that of the cone anvil 59. The inner geometry of the cone anvil 59 is generally tapered like a funnel and is pried open at the slots 61 by proximal withdrawal of the control rod 57 relative to the stationary core shaft 6, which is affixed at its distal end to the cone core support 58, which is also affixed, at is distal end, to the tapered cone core support 60. The tapered cone core 60 pries open the cone anvil so that its cutting surface 63 is larger than the diameter of the cutting edge of the cutter 8. The number of slots 61 can vary from 2 to about 20 or more and preferably ranges from about 4 to about 8.

In some embodiments, in order to facilitate withdrawal of the cone control rod 57, threads may be comprised by the rod that engage with threads in the core shaft 6 or knob 2. Rotating the anvil control knob 18 then causes the cone control rod 57 to be withdrawn proximally under mechanical advantage and considerable force and precise positional control.

The control mechanisms in the knob 2, including the control rod 57, the lock 19, the control rod knob 18, and the control rod proximal fastener 17 are illustrated as projecting axially out the proximal end of the knob 2 for simplicity of illustration. However, in this and any other embodiments illustrated within this specification, the control rod knob 18 and lock 19 preferably protrude out of a side or even the front of the knob 2 so that a rounded proximal end is presented to the user. The knob 2 can be configured similar to a gun with a handle, a trigger, and a lock mechanism all operated by the fingers of the hand while the palm of the hand pushes on the knob 2.

FIG. 10B illustrates the coring tool 56 of FIG. 10A but with the cone control rod 57 withdrawn proximally and locked by the lock 19. The cone core 58 is embedded far into the center of the cone anvil 59 and has spread the anvil sections 59 outward at the slots 61. The cutting surface 63 of the anvil 59 are properly sized to be slightly larger than the cutter 8 and thus serve as a hammer anvil with rotating cutter.

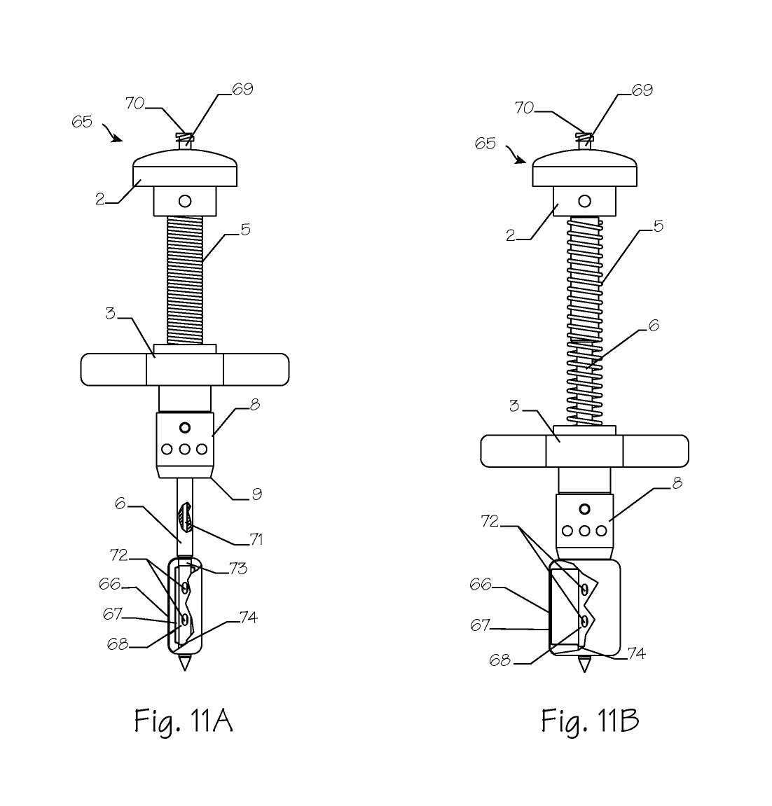

FIGS. 11A and 11B illustrate a coring tool incorporating an expandable anvil in a balloon configuration. FIG. 11A illustrates a coring tool 65 with the cutter retracted and its anvil collapsed to a first smaller diameter configuration, the coring tool 65 comprising a balloon anvil. The coring tool 65 further comprises a cutting shield 66, which is balloon expandable. Upon expansion, the cutting shield proximal surface is enlarged to present an anvil surface in apposition to the cutter. The coring tool 65 also comprises an inflatable balloon 67, disposed within the cutting shield, and operable to force the cutting shield into an expanded configuration. The cutting shield is fixed to the core shaft at its proximal end and balloon shaft 68 at its distal end. The coring tool 65 comprises the knob 2 further comprising an inflation port 69 and a Luer lock 70, the spring 5, the handle 3, the cutter 8 further comprising the distal cutting edge 9, the core shaft 6 further comprising an inflation lumen 71. The balloon shaft includes a number of inflation ports 72 located under the balloon 67. A proximal balloon bond 73, a distal balloon bond 74 secure the balloon to the balloon shaft.

The balloon anvil 67 can comprise an elastomeric balloon structure or it can preferably comprise an inelastic, non-distensible, balloon structure such as is common in angioplasty balloons. The balloon 67 can be fabricated from materials such as, but not limited to, polyester (PET), polyimide, polyamide, silicone elastomer, polyethylene, irradiated polyethylene, or the like. The outer cutting shield 66 comprises a flexible or semi-flexible material disposed about the balloon 67 and serves as a cutting surface for the cutting edge 9 when the balloon 67 and outer cutting shield 66 are expanded. The inflation ports are cut into the balloon shaft and are operably connected to the inflation lumen, which is in turn operably connected to the inflation port 69. Liquids are infused under pressure and withdrawn from the balloon inflation port 69 to provide for balloon 67 expansion and collapse, respectively. The outer cutting shield 66, or shroud, is preferably flexible but fairly thick and capable of protecting the balloon from the cutting edge 9 as it rotates into the cutting shield 66. The cutting shield 66 can be fabricated from woven, knitted, or braided fabrics. The cutting shield 66 can be pleated, creased or folded into longitudinal folds when collapsed. The cutting shield 66 is preferably heat set in a smaller diameter configuration for insertion through an incision in the tissue to be cut.

FIG. 11B illustrates the coring tool 65 from FIG. 11A with the cutter 8 advanced against the anvil system which has been expanded to its larger, second, operational configuration. The Luer lock 70 is preferably affixed and locked to a stopcock or valve (not shown) which can be selectively and controllably opened or closed to permit retention of fluid pressure within the balloon 67 and release and removal of said fluid pressure when deflation of the balloon is desired. It is not generally necessary to deflate the balloon 67 to remove the coring tool 65 and tissue core from the patient but such deflation is an option. Preferably both the balloon 67 and the shroud 66 are furled or folded when collapsed and form smooth, rigid structures when inflated under fluid (preferably liquid) pressure.

A balloon 67 having a steep proximal edge, as shown, is preferable to a balloon 67 having a slanted or tapered proximal taper such that the cutting surface is preferably substantially perpendicular to the axis of the core shaft 6. The expanded balloon 67 forces the shroud 66 outward. It is also beneficial for the shroud 66 to be affixed, at least partially to the outside surface of the balloon 67 using weak adhesives, welds, or mechanical attachments.

The coring tools described herein generally show the cutter being advanced toward the anvil or expanded anvil. Similar devices with reverse acting mechanisms can cause the cutter to remain stationary along a longitudinal axis and the anvil or expanded anvil to be withdrawn proximally against the cutter.

Typically, the surgeon manually cores the patient's hollow organ or vessel using the coring tool 1. The coring tool 1 can alternatively, be held and manipulated by a robotic arm, endovascularly routed device such as a catheter, or a laparoscopic instrument. The laparoscopic instrument is generally placed through a sheath or trocar that has been inserted into the body through a percutaneous puncture site. In a laparoscopic embodiment, the shaft 6 is extended in length, relative to the device shown in FIG. 1A. The anvil 11 is disposed at the distal end of the shaft 6 and are within the body distal to the distal end of the sheath. Furthermore, the region between the cutter 8 and the handle 3 is correspondingly extended in length so that the rotational force can be transmitted to the cutter 8, which resides within the body while the handle 3 and knob 2 are outside the body. Thus, all operational controls are outside the body and proximal to the proximal end of the laparoscopic sheath and a pressure seal. Visual control of the cutter is accomplished using a laparoscope routed through another trocar or sheath, or it is accomplished using ultrasound, fluoroscopy, or magnetic resonance imaging. The laparoscopic device is generally rigid and flexibility is not required, although it could be advantageous to make the shaft 6 flexible to allow some curvature. The laparoscopic device cutter and anvil 11 are generally between 1 and 15 mm in diameter. The length of the shaft 6 between the handle 3 and the proximal end of the cutter 8 can range between 5 and 50-cm.

An endovascular, interventional, or endoluminal device embodiment comprises a flexible shaft 6 that is capable of being routed through a sheath into a body vessel or lumen. The coring tool in this embodiment is affixed to a catheter. A hemostasis valve, fluid-tight seal or other gasket is provided at the proximal end of the sheath to prevent loss of blood, or body fluids, or the retrograde flow of air into the body. Typical cardiovascular access sheaths known in the art of endovascular access are appropriate for this application. The cutter 8 and anvil 11 reside at the distal end of the shaft 6. The shaft 6 is a torqueable axially elongate structure that also has column strength. The region between the handle 3 and the cutter 8 is generally very long in this embodiment. This length and the corresponding length of the shaft 6 may range from 10-cm to over 200-cm depending on the distance between the access site and the treatment site. The diameter of the cutter 8 is small enough to fit through the sheath, generally less than 24 French, or 8 mm in diameter. The cutter 8 and the anvil 11 can also be fabricated from structures that are radially expandable to allow them to fit through small diameter sheaths and then be enlarged to perform their coring function. The endovascular embodiment can also comprise a guidewire lumen (not shown) which is a central lumen extending from the proximal end of the knob 2 to the distal end of the device so that the device can be routed over a guidewire, a slidable fit with a lumen diameter of 0.010 inches to 0.042 inches. All rotational operations and cutter 8 to anvil 11 closure operations are performed from the proximal end of the coring tool 1.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A coring tool comprising;
a core shaft and an anvil control shaft, said anvil control shaft being longitudinally translatable relative to the core shaft;
a cutter disposed on the core shaft, said cutter being longitudinally translatable relative to the core shaft;
an expandable anvil disposed on the core shaft, distal to the cutter, said anvil having a proximal end longitudinally fixed to core shaft and a distal end longitudinally fixed to the anvil control shaft;
said expandable anvil having a small diameter configuration for insertion into a hollow body organ, and a large diameter configuration comprising a proximally facing surface opposing the cutter.

2. The coring tool of claim 1 wherein the expandable anvil comprises an expandable sleeve comprising a plurality of struts which bend radially outwardly when compressed longitudinally, to create one or more proximally facing surfaces in apposition to the cutter.

3. The coring tool of claim 1 wherein the expandable anvil comprises an expandable mesh basket, said expandable mesh basket being configurable upon longitudinal compression to provide a proximally facing surface in apposition to the cutter.

4. The coring tool of claim 1 wherein the expandable anvil comprises an expandable mesh basket, said expandable mesh basket being configurable upon longitudinal compression to provide a proximally facing surface in apposition to the cutter.

5. The coring tool of claim 1 wherein the expandable anvil comprises an expandable sleeve comprising a plurality of struts which bend radially outwardly when compressed longitudinally, to create one or more proximally facing surfaces in apposition to the cutter, and further comprising bands connecting adjacent struts.

6. The coring tool of claim 1 wherein the expandable anvil comprises an expandable sleeve comprising a plurality of struts which bend radially outwardly when compressed longitudinally, to create one or more proximally facing surfaces in apposition to the cutter, and further comprising bands connecting adjacent struts.

7. The coring tool of claim 1 wherein the expandable anvil comprises an umbrella comprising a plurality of struts supporting a membrane, said struts connected to the anvil control rod such that longitudinal movement of the anvil control rod causes the umbrella to open and create a proximally facing surface in apposition to the cutter.

8. The coring tool of claim 1 wherein the expandable anvil comprises an expandable cone on the anvil control shaft, and a cone spreader disposed on the core shaft, such that longitudinal movement of the anvil control shaft causes the spreader to force the cone toward a large diameter configuration such that the base of the cone expands to provide a proximally facing surface in apposition to the cutter.

9. The coring tool of claim 1 wherein the cutter is rotatable relative to the anvil.

10. The coring tool of claim 1 further comprising a penetrating distal tip, dispose distal to the anvil, said penetrating tip adapted to pierce tissue of the hollow body organ without first making a pilot incision in the tissue of the hollow body organ.

11. A coring tool comprising;
a core shaft;
a cutter disposed on the core shaft, said cutter being longitudinally translatable relative to the core shaft;
an configurable anvil disposed on the core shaft, distal to the cutter, said anvil having a proximal end longitudinally fixed to core shaft;
said configurable anvil having a low profile configuration for insertion into a hollow body organ, and a large profile configuration comprising a proximally facing surface opposing the cutter.

12. The coring tool of claim 12 wherein the configurable anvil comprises an expandable sleeve comprising a plurality of struts which bend radially outwardly when compressed longitudinally, to create one or more proximally facing surfaces in apposition to the cutter.

13. The coring tool of claim 11 wherein the configurable anvil comprises an expandable mesh basket, said expandable mesh basket being configurable upon longitudinal compression to provide a proximally facing surface in apposition to the cutter.

14. The coring tool of claim 11 wherein the configurable anvil comprises an expandable mesh basket, said expandable mesh basket being configurable upon longitudinal compression to provide a proximally facing surface in apposition to the cutter.

15. The coring tool of claim 11 wherein the configurable anvil comprises an expandable sleeve comprising a plurality of struts which bend radially outwardly when compressed longitudinally, to create one or more proximally facing surfaces in apposition to the cutter, and further comprising bands connecting adjacent struts.

16. The coring tool of claim 11 wherein the configurable anvil comprises an expandable sleeve comprising a plurality of struts which bend radially outwardly when compressed longitudinally, to create one or more proximally facing surfaces in apposition to the cutter, and further comprising bands connecting adjacent struts.

17. The coring tool of claim 11 wherein the configurable anvil comprises an umbrella comprising a plurality of struts supporting a membrane, said struts connected to the anvil control rod such that longitudinal movement of the anvil control rod causes the umbrella to open and create a proximally facing surface in apposition to the cutter.

18. The coring tool of claim 11 wherein the configurable anvil comprises an expandable cone on the anvil control shaft, and a cone spreader disposed on the core shaft, such that longitudinal movement of the anvil control shaft causes the spreader to force the cone toward a large diameter configuration such that the base of the cone expands to provide a proximally facing surface in apposition to the cutter.

19. The coring tool of claim 11 wherein the configurable anvil comprises a plate rotatably disposed on the anvil control shaft.

20. The coring tool of claim 11 wherein the configurable anvil comprises a balloon expandable cutting shield.

21. The coring tool of claim 11 further comprising a penetrating distal tip, dispose distal to the anvil, said penetrating tip adapted to pierce tissue of the hollow body organ without first making a pilot incision in the tissue of the hollow body organ.
